(19) United States
(12) Patent Application Publication
(10) Pub. No.: US 2011/0039784 A1
(43) Pub. Date: Feb. 17, 2011

(54) COMPOSITION FOR TREATMENT OF ERECTILE DYSFUNCTION

(75) Inventors: Sung Won Lee, Seoul (KR); Mee Ree Chae, Seoul (KR); In Suk So, Seoul (KR)

Correspondence Address: KNOBBE MARTENS OLSON & BEAR LLP, 2040 MAIN STREET, FOURTEENTH FLOOR, IRVINE, CA, 92614, US

(73) Assignees: SUNGKYUNKWAN UNIVERSITY Foundation for Corporate Collaboration, SUWON-SI (KR); SAMSUNG LIFE WELFARE FOUNDATION, SEOUL (KR)

(21) Appl. No.: 12/605,084

(22) Filed: Oct. 23, 2009

(30) Foreign Application Priority Data
Aug. 12, 2009 (KR) 10-2009-0074178

(51) Int. Cl. A61K 38/00; A61K 31/7088

(52) U.S. Cl. 514/17.4; 514/44 R; 514/355

(57) ABSTRACT

Disclosed herein is a therapeutic composition for erectile dysfunction comprising as an active ingredient a gene functioning to inhibit expression of an ion channel involved in influx of ions into a tissue. When expressed, the gene inhibits the activity of the ion channel to effectively block calcium influx. Accordingly, the composition can be effective for treating erectile dysfunction without side effects.

### BACKGROUND OF THE INVENTION

1. Field of the Invention

The present invention relates to a therapeutic composition for erectile dysfunction. More particularly, the present invention relates to a composition for the treatment of erectile dysfunction comprising as an active ingredient a gene functioning to inhibit the expression of an ion channel involved in the influx of ions into a tissue.

2. Description of the Related Art

Erectile dysfunction is one of the main diseases threatening the physical and mental health of adults, having a prevalence rate higher than that of any other adult disease known so far. Erectile dysfunction is so common as to be found in as many as 10% of adult males, affecting about 150 million adult males all over the world. In Korea, 2.5-3 million men were reported to suffer from the disease. Erectile dysfunction is not a disease that is deadly, but is one of the most important factors determining the ‘quality of life’.

With the rapid growth of an elderly population, and the rapid growth of stress and overwork attributed to rapid industrial development, patients suffering from erectile dysfunction have been sharply increasing in number. It is expected that patients suffering from erectile dysfunction will reach 300 million in 2020, which is twice as many as the number of patients today.

The modulation of the contractility of corporal smooth muscle is critical in achieving penile erection. The impaired relaxation of corporal smooth muscle resulting from heightened contractility thereof is directly correlated with the pathophysiology of erectile dysfunction. Corporal smooth muscle tone is modulated through extracellular signals, i.e. by various neurotransmitters in the sympathetic and parasympathetic nervous systems where potassium channels and calcium channels play a critical role in the contraction and relaxation of cells. In various types of smooth muscle including vascular smooth muscle, non-adrenergic cholinergic substances are known to regulate the opening of potassium and calcium ion channels through intracellular secondary neurotransmitters whereby the intracellular level of calcium ions can be changed to regulate the contractility of cells (Christ et al., 1995). A potassium ion channel has the cytophysiological function of allowing the efflux of lots of potassium ions, that is, cations when opened, to cause a negative membrane potential to exist on the cell. When shifted from depolarization to hyperpolarization through the above-mentioned mechanism, the cell has such a stable membrane potential as to close the voltage-dependent calcium channel, thereby reducing the intracellular calcium ion level to cause the relaxation of the cell. As such, the potassium ion channels and the voltage-dependent calcium ion channels regulate the contractility of smooth muscle through actions which are antagonistic to each other. Also, potassium ion channels function to maintain stable membrane potentials to regulate the arrival of a threshold for depolarization, thereby playing an important role in the relaxation of cells. Thus, the aberration of potassium ion channels does not allow the maintenance of normal stable membrane potentials, so that not only do the cells easily reach a threshold, giving rise to the contraction of smooth muscle, but also that repolarization required for relaxation is delayed in the contracted cells, making the contraction last longer and heightening the contractility of the smooth muscle tissue. The impairment of potassium ion channels in corporal smooth muscle is directly correlated with the pathophysiology of erectile dysfunction.

Except for voltage-dependent calcium channels, there are two additional kinds of calcium channels that regulate intracellular calcium levels in smooth muscle: Receptor Operated Calcium Channel (ROCC) activated when G-protein coupled receptor (GPCR) is activated, and Store Operated Calcium Channel (SOC) activated when intracellular calcium is depleted. Extensive studies have been extensively done on such calcium channels in vascular smooth muscle, revealing that these ligand-gated calcium channels play physiologically more important roles than do the voltage-dependent calcium channels (Welsh et al, 2002). The reason for paying lots of attention to ROCC or SOC is that many experiments are being conducted all over the world using genes coding for transient receptor potential (TRP) channels as they are suggested as molecular counterparts to ROCC and SOC, which has until now only been able to be presumed. It has been known that when a neurotransmitter acts on smooth muscle, intracellular calcium levels increase because of two types of behavior. First, the intracellular calcium level transiently increases, followed by the maintenance of calcium influx. Keen attention is paid to ROCC and SOC as calcium sources which act in the later stages. ROCC is a non-selective cation channel with a calcium permeability up to 10 times higher than monovalent permeability. In contrast, SOC is highly selective for calcium ions and has been named calcium release activated calcium channel (CRAC) by some researchers because it is activated when intracellular calcium is released. There are TRP channels corresponding to respective calcium channels. Although different according to scientists, counterparts are generally suggested to be TPRC6 for ROCC (Inoue et al., 2001; Jung et al., 2002; Welsh et al., 2002) and TRPC1, TRPC4 and TRPV6 (CaT1, ECaC2, CaT-L) for SOC or CRAC (Xu & Beech, 2001).

Studies on the physiology of penile erection focus on intra- and extracellular neurotransmitters involved in the regulation of the contractility of corporal smooth muscle. Recent studies have revealed that the TRPC6 channel plays various roles and physiological functions as concerns the association with neurotransmitters in modulating the tension of cells. However, nowhere has the mechanism of operation of TRPC6 channels in corporal smooth muscle been mentioned in previous studies. In this study, TRPC6 ion channels are found to play a physiological role as calcium sources in human corporal smooth muscle. In practice, after in vivo transfection of the cDNA of the TRPC6 dominant negative form thereinto, diabetic rats were examined for a change in the intracavernosal pressure of the corpus cavernosum and for effect on the modulation of smooth muscle tone.

### SUMMARY OF THE INVENTION

It is therefore an object of the present invention to provide a therapeutic composition for erectile dysfunction which is significantly improved in safety and effectiveness as compared to conventional therapeutic agents.

In accordance with an aspect thereof, the present invention provides a therapeutic composition for erectile dysfunction, comprising as an active ingredient a gene functioning to inhibit expression of an ion channel involved in influx of ions into a tissue.

So long as it belongs to an animal, any tissue may be employed in the present invention. Preferably, it is corporal smooth muscle. In an embodiment of the present invention, the ions are calcium ions (Ca²⁺).

So long as it is involved in the influx of ions into a specific tissue, any ion channel may be used in the present invention. Preferably, it is a TRPC6 ion channel.

No limitations are imparted to the gene if it is a dominant negative form of the ion channel. Preferably, the gene has a nucleotide sequence of SEQ ID NO. 1.

When expressed, the gene inhibits the activity of the ion channel to effectively block calcium influx into cells. Thus, the composition is useful in the treatment of erectile dysfunction without side effects.

### BRIEF DESCRIPTION OF THE DRAWINGS

The above and other objects, features and advantages of the present invention will be more clearly understood from the following detailed description taken in conjunction with the accompanying drawings, in which:

FIG. 1 shows the comparison of partial nucleotide sequences between wild-type TRPC6 and mutant TRPC6^{DN}.

FIG. 2 is of fluorescent confocal images of HEK 293 cells (A, Scale bars=10 μm) and corporal smooth muscle cells (B, Scale bars=50 μm) transiently transfected with pDNAs for ion channels rslo, K_{ATP} (Kir 6.2 plus SUR2B), TRPC6 and TRPC6^{DN}.

FIG. 3 is of electrophysiological properties of human embryonic kidney (HEK 293) cells transfected with wild-type TRPC6, showing a representative current trace of TRPC6 channels at a holding potential of −60 mV (A), current-voltage (I-V) relationship of TRPC6 currents obtained during voltage ramps ranging from −100 mV to +100 mV (B), current-voltage of 200 μM phenylephrine (PE) and 100 μM carbachol (CCh) induced currents, obtained after leak subtraction (C), and summary of the mean±SD of whole-cell currents at −100 mV and +100 mV (n=5) (D);

FIG. 4 is of electrophysiological properties of human corpus cavernosum (HCC) cells transfected with wild-type TRPC6, showing representative whole-cell currents at a holding potential of −60 mV with the external solution containing 140 mM Cs, and 100 nM of added ET-1 (A) and current-voltage (I-V) relationship obtained during voltage ramps ranging from −100 mV to +100 mV (B);

FIG. 5 is of electrophysiological properties of human corpus cavernosum (HCC) cells transfected with dominant negative TRPC6, showing a representative current trace at a holding potential of −60 mV (A) and current-voltage (I-V) relationship of the same cell obtained during voltage ramps ranging from −100 mV to +100 mV (B);

FIG. 6 is a graph showing a summary of the means±SD of peak whole-cell currents from pDNA/wild-type or dominant negative TRPC6-transfected cells at +100 mV and −100 mV.

FIG. 7 is of electrophysiological properties of corporal smooth muscle cells, showing an ET-1-induced representative intracellular calcium level trace in control (A), in TRPC6-transfected cells (B), and in dominant negative TRPC6-transfected cells, performed in physiological salt solution (n=5) (C) and a comparison of the results obtained in (C) (D);

FIG. 8 is of electrophysiological properties of rslo-transfected HEK 293 cells, with currents obtained during voltage ramps ranging from −100 mV to +80 mV in an extracellular solution comprising 135 mM NaCl, 5 mM KCl, 1 mM MgCl₂, 2 mM CaCl₂, 10 mM HEPES, 5 mM Glucose, pH 7.4 and a pipette solution comprising 140 mM KCl, 2 mM MgCl₂, 5 mM EGTA, 10 mM HEPE, 5 mM K₂-ATP, pH 7.2 at pCa 6.5 (A) and pCa 7 (B);

FIG. 9 is of electrophysiological properties of rslo-transfected HCC cells, with currents obtained during voltage ramps ranging from −100 mV to +80 mV in an extracellular solution comprising 135 mM NaCl, 5 mM KCl, 1 mM MgCl₂, 2 mM CaCl₂, 10 mM HEPES, 5 mM Glucose, pH 7.4 and a pipette solution comprising 140 mM KCl, 2 mM MgCl₂, 5 mM EGTA, 10 mM HEPE, 5 mM K₂-ATP, pH 7.2 at pCa 6.5 (A) and pCa 7 (B);

FIG. 10 is of graphs showing a summary of the means±SD of currents at +60 mV.

FIG. 11A shows representative traces of ATP-induced intracellular calcium level ([Ca²⁺]ᵢ) responses in HEK 293 cells transfected with pDNAs/ion channel (solid lines) or pEGFP vector only (control; dotted lines): (a) rslo, (b) rslo+TRPC6^{DN} (c) K_{ATP}: (Kir 6.2+SUR2B), (d) TRPC6^{DN} (e) TRPC6^{DN}+K_{ATPd}. FIG. 12B shows a summary of [Ca²⁺]ᵢ levels caused by ATP in bar graphs. Each column represents mean±SD. The experiments were performed in a physiological salt solution.

FIG. 12 shows the cytotoxicity of expression of ion channels in membrane damaging effects determined by LDH assay. The release of the cytoplasmic lactate dehydrogenase of HEK cells was quantified at 0 hr, 24 hr, 48 hr and 72 hr after transfection of pcDNA. Results are shown as mean±SD of three experiments.

FIG. 13 is semi-quantitative RT-PCR analysis of TRPC6 mRNA levels in cavernosal tissue of age-matched control (normal) and 16 weeks STZ-induced diabetic rats. Normal Rat: lane 1˜3, Diabetic Rats: lane 4˜6 (A). For quantitative analysis, density of each DNA band was measured by densitometry (B).

FIG. 14 shows representative time course of changes in intracavernosal pressure (ICP)/arterial blood pressures (BP) from electric field stimulation on corpus cavernosum in young control rats (4 month) (A), diabetic rats transfected with pcDNA vector only (4 month) (B), and diabetic rats transfected with TRPC6^{DN} (C) and shows a summary of ICP and BP data in all experimental groups (D). In vivo experiments were performed 14 days after transfection with the cDNA pcDNA in rats. Data are represented as mean±SD.

FIG. 15 shows RT-PCR detection of mRNA expression (537 bp) of the dominant negative form of TRPC6 in diabetic rat cavernous tissue 7 days after gene transfer. Lane 1: negative control (without RT), lane 2: transfected with vector only (control), lane 3: transfected with pcDNA of TRPC6 dominant negative form.

### DESCRIPTION OF THE PREFERRED EMBODIMENTS

A better understanding of the present invention may be obtained through the following examples which are set forth to illustrate, but are not to be construed to limit the present invention.

### Example 1

### Cloning of a Dominant Negative form of TRPC6 Known as Molecular Counterpart of ROCC

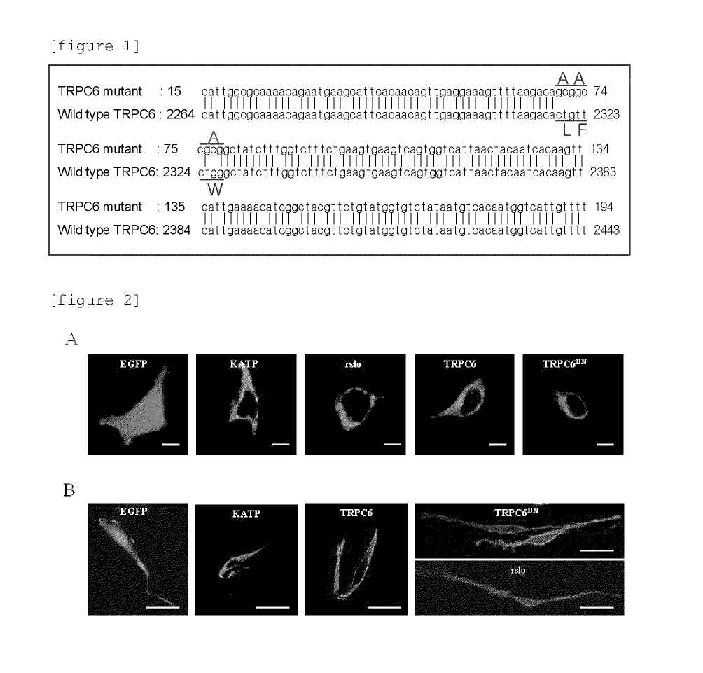

A dominant negative form of TRPC6 (SEQ ID NO. 1) was prepared through the mutation of three amino acid residues (L678˜W680) in the pore region of wild type TRPC6 (SEQ ID NO. 2) into alanine residues using QuikChang Site-Directed Mutagenesis kit (Stratagene). To this end, two complementary oligonucleotide sequences were used as primers for the amplification of a mutant TRPC6 with a plasmid DNA serving as a template. After the wild-type gene was excised with the restriction enzyme Dpn I, the mutant DNA was transformed into an E. coli host cell to obtain a mutant clone which was subjected to DNA sequencing (FIG. 1).

### Example 2

### Expression Pattern of Ion Channel Genes in Cells

The plasmid pcDNA carrying the ion channel genes rslo, K_{ATP}, TRPC6 or TRPC6^{DN} was cotransfected together with pEGFP into cells with the aid of lipofectamine. After 24 hours of incubation, the cells were monitored for the expression pattern of the genes under a confocal laser microscope. When transformed with pEGFP vector alone, the cells showed uniform fluorescence over the cytoplasm and nucleus. In contrast, fluorescence was observed mainly on the plasma membrane when pGFP and the pDNA of each ion channel were transfected together. These results showed a gene of interest can be overexpressed or inhibited from being expressed, giving basic data on the basis of which the physiological activity of corporal smooth muscle can be monitored (FIG. 2).

### Example 3

### Electrophysiological Change of a TRP Channel in Human Corporal Smooth Muscle Cells In Vitro Transfected with a Dominant Negative Form of TRP Channel

(1) Electrophysiological Change in Human Embryonic Kidney (HEK 293) Cell Transfected with Wild-type TRPC6

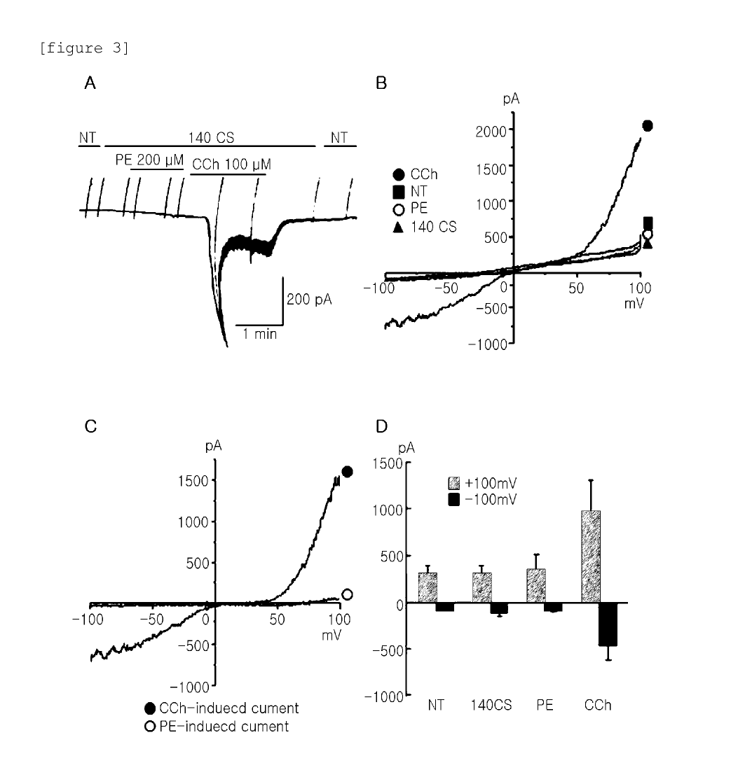

The physiological function of the TRPC6 ion channel was examined in HEK cells which are free of ion channels. In this regard, GFP-TRPC6 pcDNA was transfected into HEK cells using a liposome and expressed therein. After the membrane potential was held at −60 mV, whole-cell currents were recorded in symmetrical 140 mM Cs conditions. Upon the application of 200 μM PE (phenylephrine), nearly no currents changed. However, treatment with 100 μM CCh (carbachol) increased inward currents (FIG. 3A). At −100 mV, the current was −112.0±18.1 pA (n=5) in a 140 mM extracellular Cs condition, −90.0±15.0 pA (n=5) after adding 200 μM PE, and −460±169.5 pA (n=5) after adding 100 μM CCh. The current-voltage (I-V) relationship of TRPC6 was linear during voltage ramps ranging from −100 mV to +100 mV for 1.8 sec and showed outward rectification above +50 mV (FIG. 3B). The PE/CCh-induced current was obtained after leak subtraction was performed (FIG. 3C).

(2) Electrophysiological Change in Human Corporal Smooth Muscle Cells Transfected with Wild-Type TRPC6

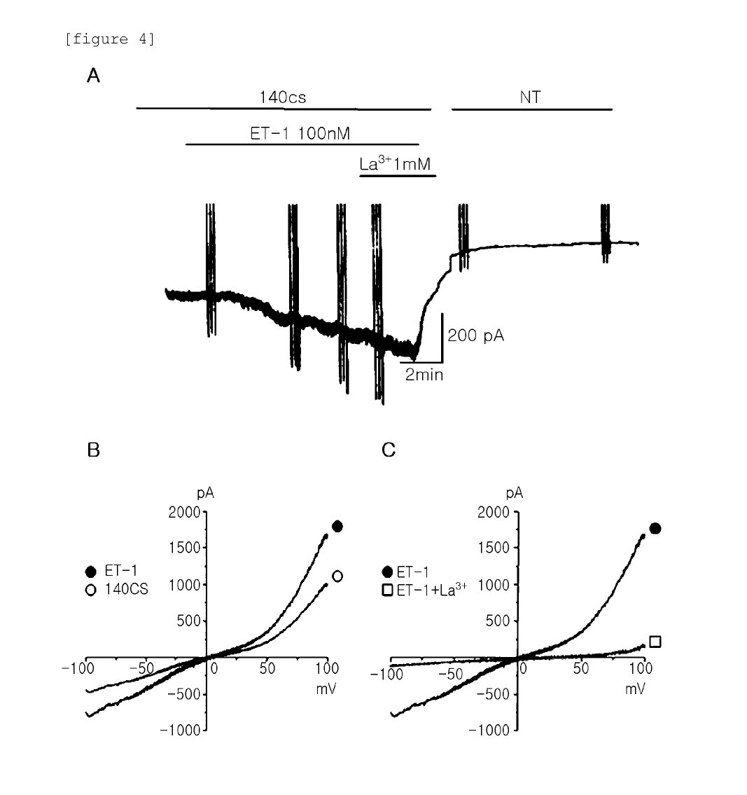

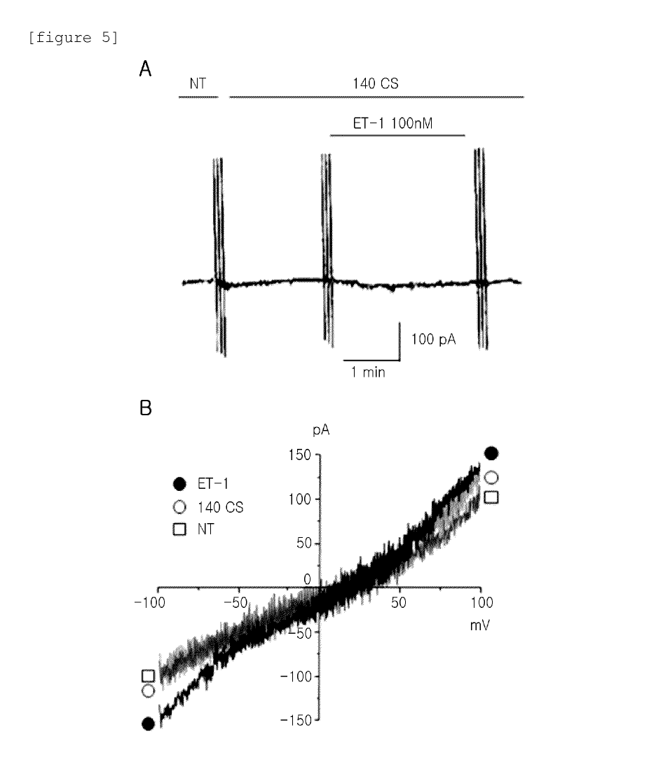

As described with HEK cells, the wild-type TRPC6 cDNA was expressed. After being selected, green-fluorescent cells were examined for the activity of the ion channel using a whole-cell patch clamp technique. At the −60 mV holding membrane potential with the external solution changed from Na-rich normal tyrode solution to 140 mM Cs, the inward current was increased; whereas no currents changed upon external treatment with ET-1, a typical inducer of smooth muscle contraction. At −100 mV, the current was −535.9±162.4 pA (n=5) in 140 mM extracellular Cs, and −462.1±150.5 pA (n=5) after adding 100 nM ET-1. The current-voltage (I-V) relationship of corporal smooth muscle cells was similar to that of HEK cells activated by CCh. The inward current was also increased (at −100 mV, −539.5±116.4 pA, n=3) in 140 mM Cs, and further increased to −1559.7±532.5 pA (n=3) at −100 mV when adding ET-1. The current was completely decreased after adding 1 mM La³⁺. (FIG. 4)

(3) Electrophysiological Change After Expression of Dominant Negative Form of TRPC6 in Human Corporal Smooth Muscle

For genetic regulation of the TRPC6 ion channel in corporal smooth muscle cells, the cells transfected with the dominant negative form of the TRPC6 ion channel were examined for electrophysiological function. The current showed no change compared to the control after the dominant negative form of TRPC6 expressed in corporal smooth muscle cells (FIG. 5).

At −100 mV, the current was −206.7±130.2 pA (n=3) in 140 mMCs, and −259.6±127.8 pA (n=3) after adding 100 nM ET-1.

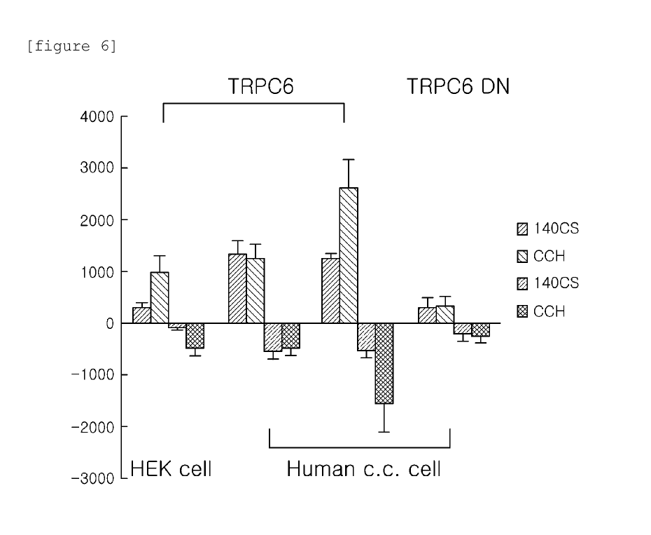

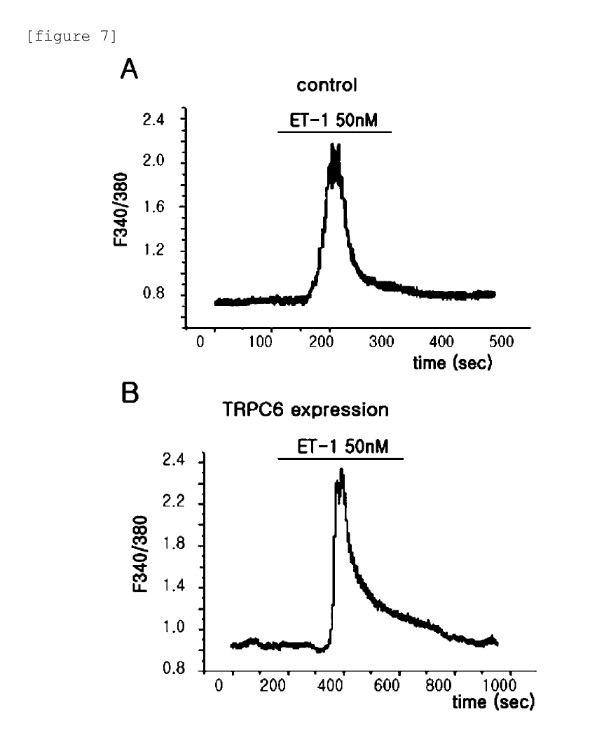

As described above, the electrophysiological changes of corporal smooth muscle cells after the expression of TRPC cDNA (wild-type and dominant negative form of TRPC6) were revealed for the first time. This patch clamp study indicates that the dominant negative form of TRPC6 may be a candidate for use in gene therapy for erectile dysfunction through the regulation of ion channels in corporal smooth muscle (FIG. 6).

### Example 4

### Effect of Transfection with Dominant Negative Form of TRPC6 on Intracellular Free Ca²⁺ in Corporal Smooth Muscle

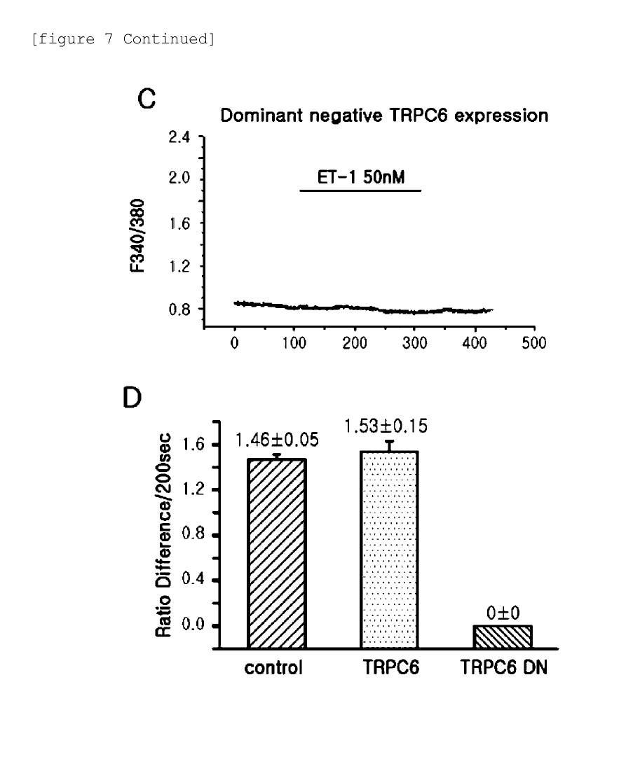

After fura-2 was loaded thereto, cultured corporal smooth muscle cells were measured for intracellular calcium levels. The extracellular addition of 50 nM endothelin-1 induced an increase in intracellular calcium level, with the separation of an initial increasing phase and a late constant phase (FIG. 7A). Intracellular calcium levels were observed to undergo similar changes between the corporal smooth muscle cells transfected with TRPC6 cDNA and the control cells (FIG. 7B).

Between the TRPC6 cDNA-transfected cells and the control cells, there are little differences in the initial phase. The second phase which remained constant also showed no significant differences although some cell increased in the second phase. Intracellular calcium levels were also measured in the corporal smooth muscle cells where the dominant negative form of TRPC6 was expressed. The addition of ET-1 induced no changes in intracellular calcium level (FIG. 7C). Collectively, F340/380, accounting for ET-1-induced calcium increase, was measured to be 1.47±0.03 (n=3) in corporal smooth muscle cells (control), 1.53±0.09 (n=3) in corporal smooth muscle cells where TRPC6 was overexpressed, and 0±0 (n=3) in corporal smooth muscle cells where the dominant negative form of TRPC6 was expressed (FIG. 7D). The initial phase, known to be responsible for the calcium increase resulting from IP3-induced calcium release from intracellular sources, was suppressed by the expression of the dominant negative form of TRPC6, indirectly indicating that TRPC channels function to supply calcium to the intracellular calcium sources as reported previously.

### Example 5

### Electrophysiological Change after In Vitro Transfection with K_{ca}, Channel Gene

(1) Expression of rslo Gene in Human Embryonic Kidney (HEK 293) Cell

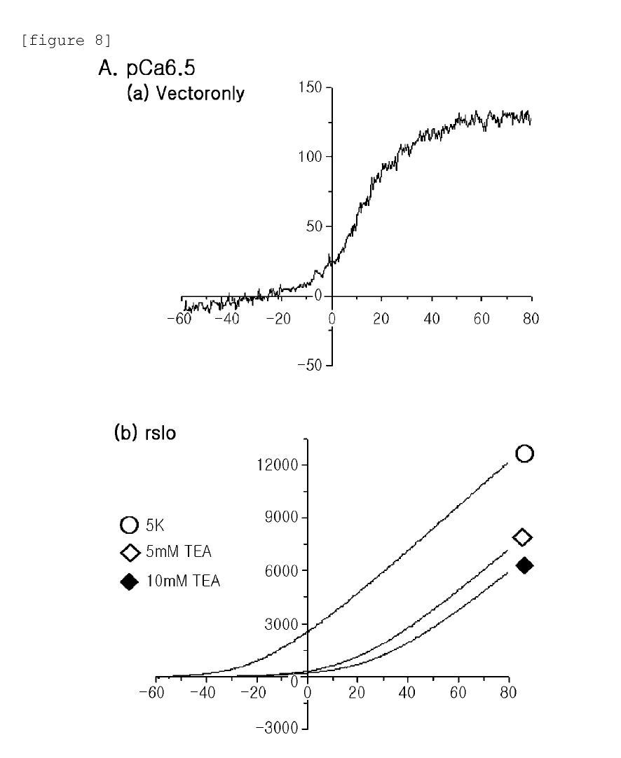

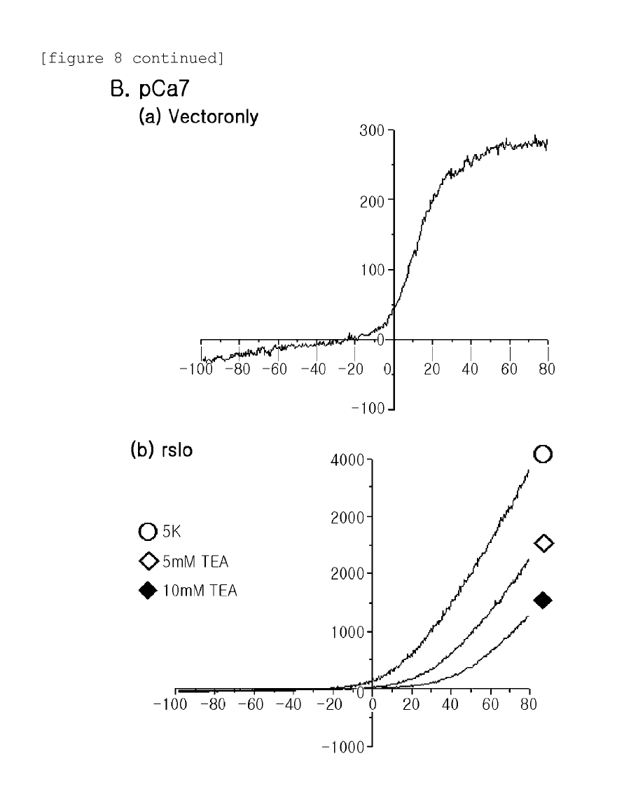

The maxi-K ion channel is known to play an important role in reducing the contraction of smooth muscle responsible for the modulation of penile erection and relaxation. Together with GFP, the α-subunit (rSlo) coding sequence of calcium-dependent potassium ion channel cloned from the rat brain was subcloned into pcDNA3.1(+). After the expression of rSlo gene in HEK cells, green-fluorescent cells were selected under a mercury lamp light and observed for the electrophysiological properties of the ion channels using a whole-cell patch clamp technique. Cells were used within 24˜48 hrs after expression. When an infusion solution and a pipette solution were respectively 5 and 140 mM KCl with the intracellular calcium level fixed at pCa 6.5, the current was increased to 356.2±28.2 pA/pF (at +60 mV, n=8) in the rslo-expressed cells, which showed an outward current 65 times as large as that of the control (+60 mV, 5.5±0.5 pA/pF n=8) (FIG. 8A). At an intracellular calcium level of pCa 7, the current was read 10.4±3.2 pA/pF (+60 mV, n=6) in the control and 100.5±15.4 pA/pF (n=6) in the rslo-expressed cells (FIG. 8B). The outward current was decreased in a dose-dependent manner by TEA, a selective blocker of the maxi-K⁺ ion channel. The current-voltage (I-V) relationship showed membrane potential-dependent outward rectification characteristic of the maxi-K ion channel, and was dependent on intracellular calcium levels.

(2) Expression of rslo Gene in Human Corporal Smooth Muscle Cell

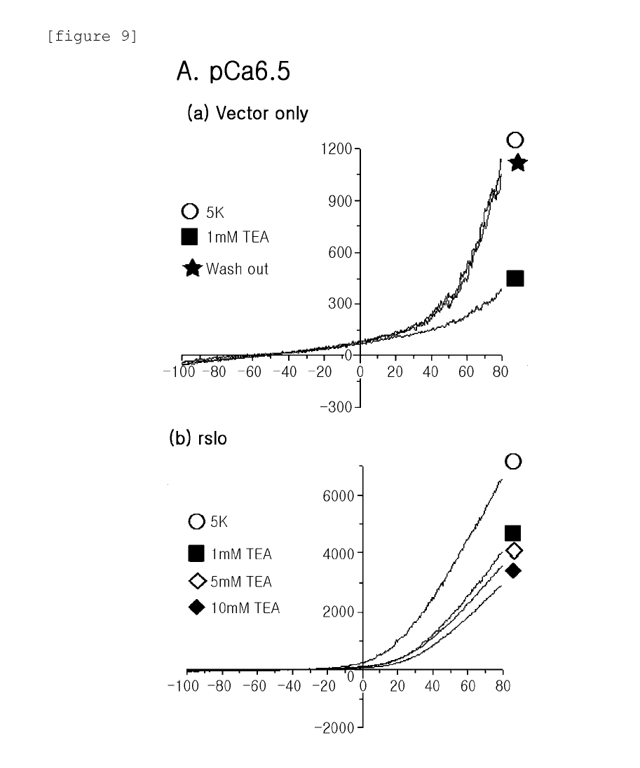

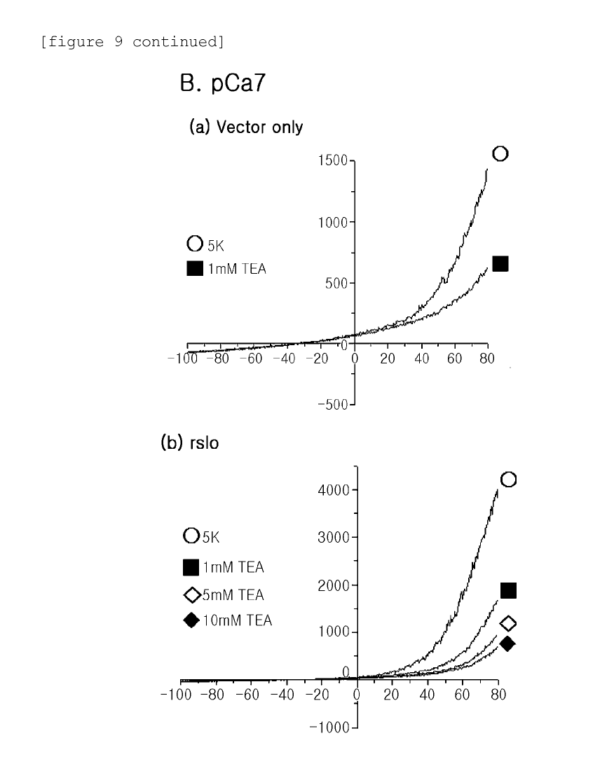

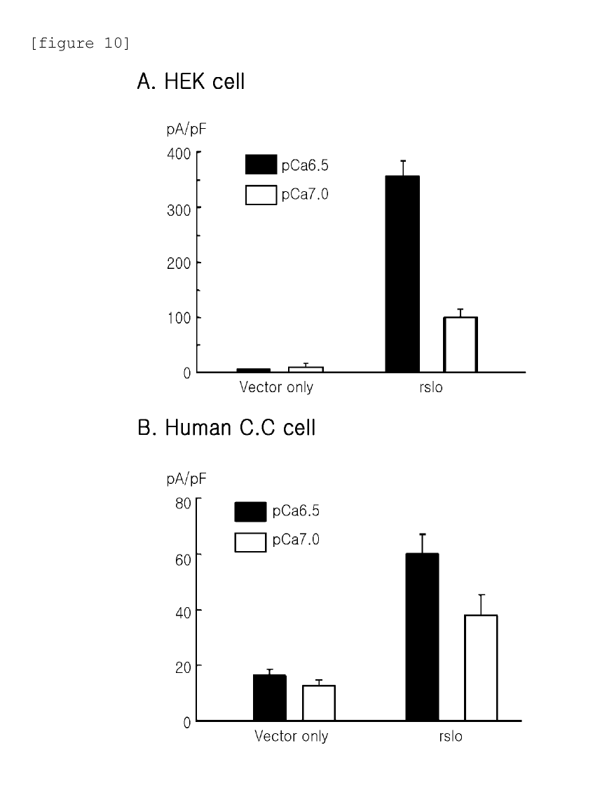

As described with HEK cells, the rslo pcDNA was expressed. The ion channels were examined for activity using a whole-cell patch clamp technique. Under the condition of a pipette solution having a calcium concentration of pCa 6.5, the current was 60.1±7.2 pA/pF (n=22) at a membrane potential of +60 mV in the rslo-expressed cells, which increased in outward current four-fold compared to that of control (16.4±2 pA/pF n=9) (FIG. 9A). Under the condition of an intracellular calcium concentration of pCa 7, the current was 12.8±2 pA/pF (+60 mV, n=9) in the control and 38.1±7 pA/pF (+60 mV, n=13) in the rslo-expressed cells (FIG. 9B). The outward current was decreased in a dose-dependent manner by TEA, a selective blocker of the maxi-K⁺ ion channel. The current-voltage (I-V) relationship was similar to that recorded in the HEK cells. This experiment demonstrated that the transferred gene of the rslo ion channel was functionally active in human corporal smooth muscle cells (FIG. 10).

### Example 6

### Effect of Ion Channel Gene Transfer on Intracellular Free Calcium Level and Combination of Ion Channels for Optimal Regulation

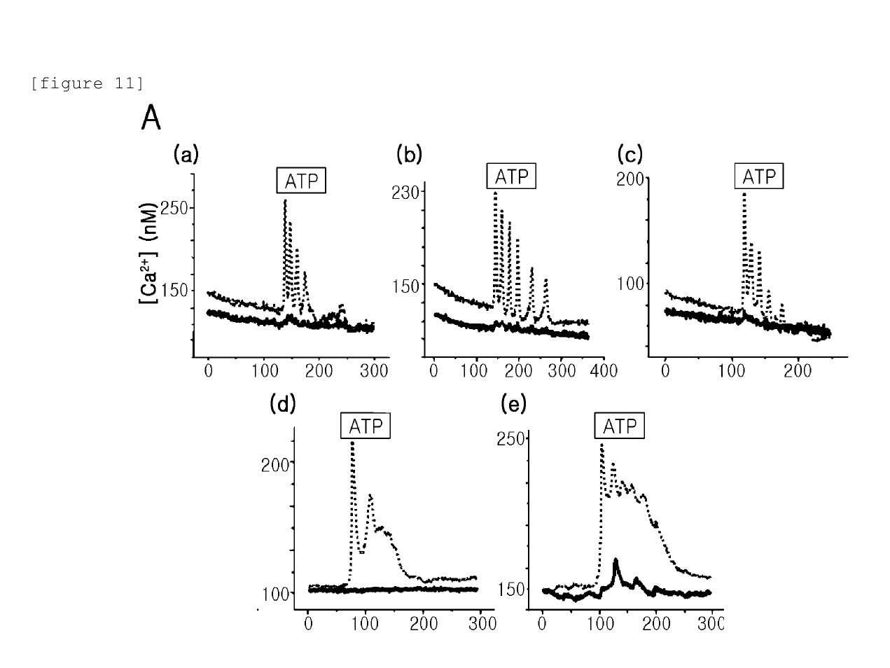

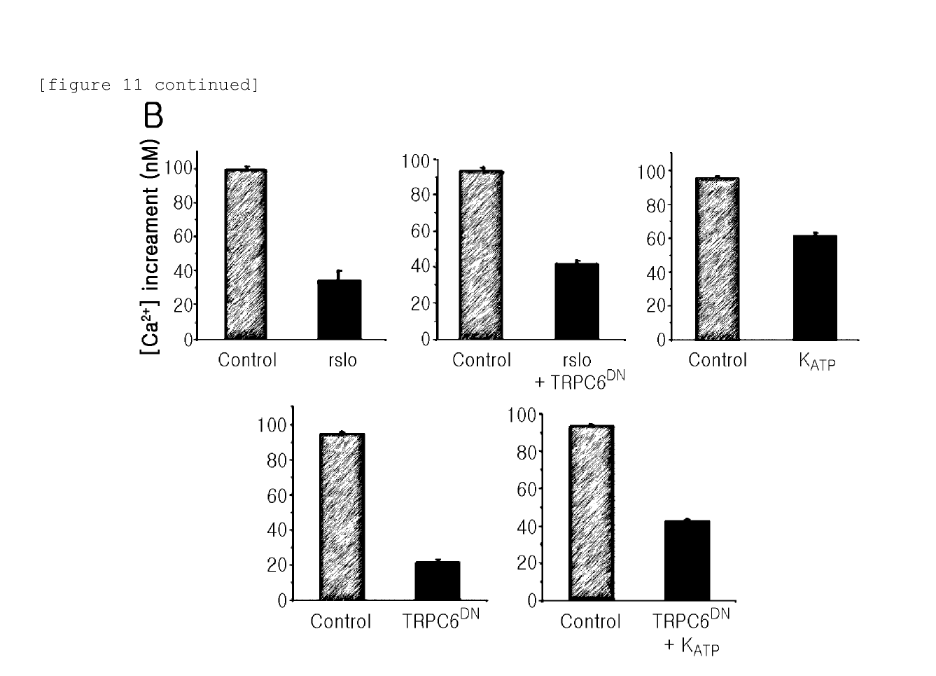

In order to investigate the effect of gene transfer on intracellular calcium level, the most important factor for smooth muscle contraction, ion channel genes were expressed separately or in combination in HEK cells, followed by treatment with ATP. There were no significant changes in basal calcium level, compared to the control whereas ATP effectively induced the intracellular calcium levels to be reduced in ion channel-expressed cells (rslo: 66.2±5.8%, n=10; rslo+TRPC6^{DN}: 55.5±1.7%, n=12; K_{ATP}36.2±2.7%, n=17; K_{ATP}+TRPC6^{DN}: 54.4±2.5%; TRPC6^{DN}: 78.2±2.1% n=10). Particularly, the gene transfer of the dominant negative form of TRPC alone was the most effective in reducing intracellular free calcium levels (FIG. 11).

### Example 7

### Evaluation for Cytotoxicity of Gene Expression through MTT and LDH Release Assay

In order to evaluate the cytotoxicity induced by the expression of a transfected gene, cell death was quantitatively analyzed by lactate dehydrogenase (LDH) release assay for cell necrosis (Sigma Co. St. Louis, Mo., USA). After HEK 293 cells were cultured in 12-well plates for 24 hr, 1 μg of the pcDNA was transfected using a FuGENE 6 transfection reagent (Roche). The release of the cytoplasmic LDH of HEK cells was quantified in the supernatants 0 hr, 24 hr, 48 hr and 72 hr after the transfection of pcDNA. The value of the LDH release assay was calculated if mean value at 0 hr was 100.

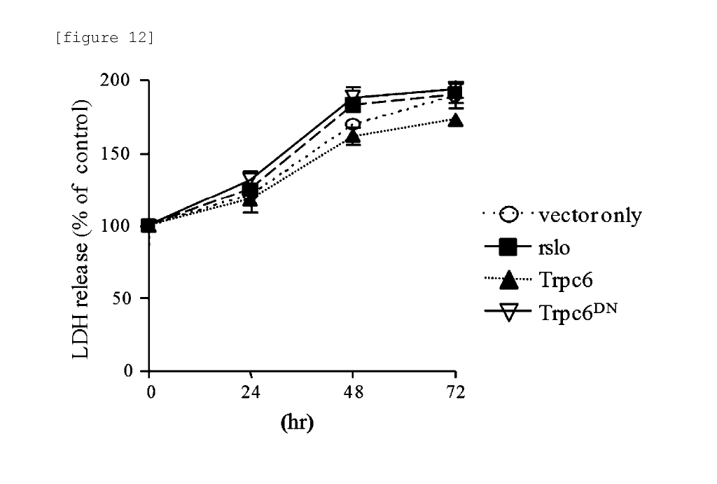

After the transfection of rslo, TRPC6 and TRPC6^{DN}, the LDH level increased with time, but was not statistically significant compared to controls transfected with the vector alone (P>0.1). At this time, each of the genes showed an expression efficiency of 80% or higher, with no difference therebetween. Taken together, the results indicated that when overexpressed, the genes cloned in the present invention did not significantly differ from the control in plasma membrane damage, cell growth and survival as proven by LDH assays (FIG. 12).

### Example 8

### Semi-Quantitative RT-PCR Analysis of TRPC6 Gene Expression in Diabetic Rats

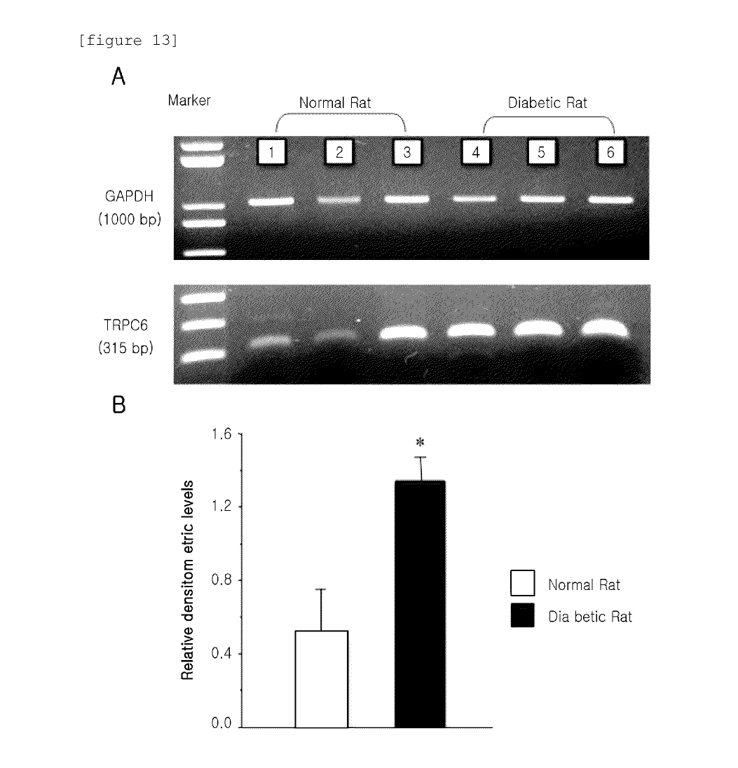

In order to determine changes in TRPC6 gene expression in diabetic corporal tissue, semiquantitative RT-PCR analyses were performed. 1.5 □g total RNA was extracted from frozen corporal smooth muscle of normal and diabetic rats using TRIzol (Invitrogen, CA, USA) method according to the manufacturer's instructions. The PCR reaction was performed with the use of I-MAX II DNA Polymerase (Intron, Korea) with the following combination of primers: TRPC6 mRNA sense primer was 5′-GTG CCA AGT CCA AAG TCC CTG C-3′ (SEQ ID NO. 3), and the antisense primer was 5′-CTG GGC CTG CAG TAC GTA TC-3′ (SEQ ID NO. 4); these reagents yield an expected product of 315 bp. The optimized PCR conditions for amplifying TRPC6 were 31 cycles at 95° C. for 1 min, 57° C. for 1 min, 72° C. for 2 min, with a final extension step at 72° C. for 10 min. The expression of GAPDH was used as an internal control for RNA input. The sense primer was 5′-ATA GAC AAG ATG GTG AAG GTC-3′ (SEQ ID NO. 5), and the antisense primer was 5′-TAC TCC TTG GAG GCC ATG TAG-3′(SEQ ID NO. 6). The optimized PCR conditions for amplifying GAPDH were 21 cycles at 95° C. for 1 min, 57° C. for 1 min, 72° C. for 2 min, with a final extension step at 72° C. for 10 min. PCR products were separated by 1.5% agarose gel electrophoresis, and the DNA bands were analyzed by densitometry. Relative gene expression levels were calculated as the ratio of the corresponding intensities of PCR product to that of GAPDH. As shown in FIG. 8, TRPC6 gene expression was increased approximately 2.7 fold in the diabetic corporal tissue compared to the normal rats The mean TRPC6/GAPDH ratio in diabetic rats was 1.34±0.14 and that for age-matched normal rats was 0.50±0.25 (p<0.05, n=3) (FIG. 13).

Based on these data and previously reported articles, we concluded that over-expressed TRPC6 channel is a good molecular candidate for treating DM-induced erectile dysfunction.

### Example 9

### Effect of TRPC6^{DN} Gene Transfer on Intracavernosal Pressure of the Corpus Cavernosum in Diabetic Rats

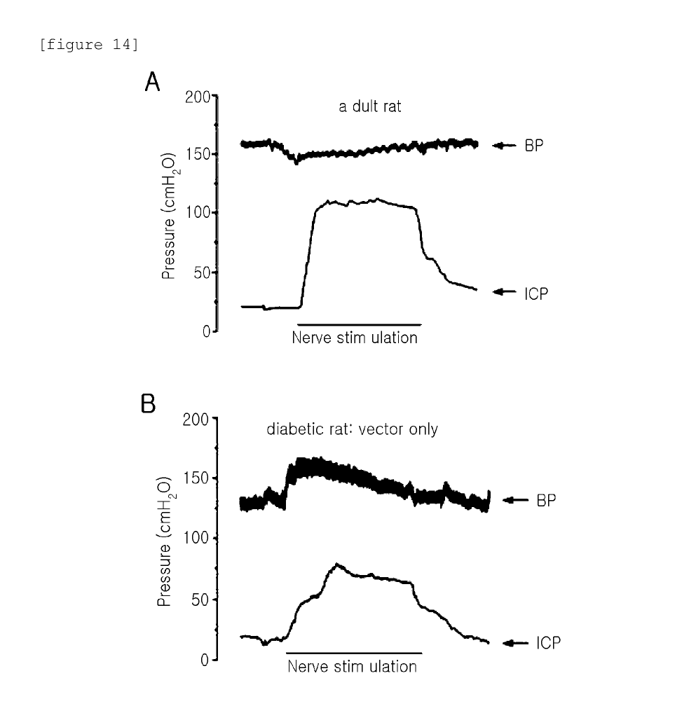

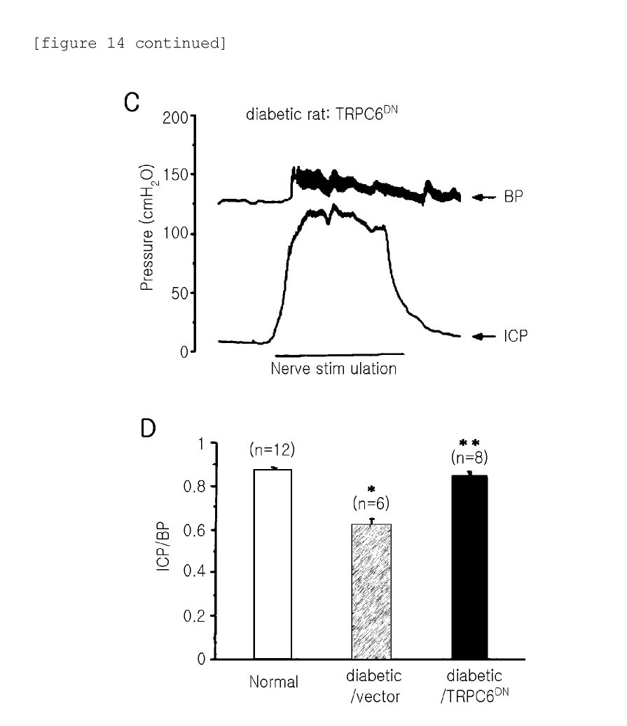

For genetic regulation of the TRPC6 ion channel involved in the contraction of corpus cavernosum, pcDNA (200 μg/100 μl) with the dominant negative form of TRPC6 was in vivo transfected into diabetic rats, followed by the examination of intracavernous pressure therein. In response to cavernosal nerve stimulation for 18 weeks, diabetic rats showed an ICP (intracavernous pressures)/BP (arterial blood pressures) ratio of 59.6±3.0% (n=8), which was significantly lower than that of non-diabetic young rats (85.8±2.6% (n=10)) (p<0.001). By contrast, diabetic rats transfected with the TRPC6^{DN} gene showed a mean ICP/BP ratio of 83.3±2.1% (n=8) and thus restored the intracavernous pressure to the normal level (p<0.001 vs diabetic rats) (FIG. 14).

### Example 10

### RT-PCR to Confirm TRPC6^{DN} Gene Expression

To evaluate the change in ICP related to TRPC6^{DN} channel gene expression, RT-PCR was used on total RNA isolated from the cavernosal tissue of the rats. RNA was used to synthesize the first strand by using Superscript III First-Strand Synthesis System, and random hexamers (Invitrogen, Carlsbad, Calif., USA). The cDNA reverse transcription product was amplified with GFP-specific primers by 2 stage nested PCR. For the first round of amplification, GFP mRNA sense primer was 5′-CTG ACC CTG AAG TTC ATC TG-3′ (SEQ ID NO. 7), and the GFP mRNA antisense primer was 5′-TAC CGT CGA CTG CAG AAT-3′ (SEQ ID NO. 8); these reagents yield an expected product of 721 bp. For the second round of amplification, the sense primer was 5′-TCG TGA CCA CCC TGA CCT A-3 (SEQ ID NO. 9) and the antisense primer was 5′-CTT GTA CAG CTC GTC CAT GC-3 (SEQ ID NO. 10); these primers lead to a 684 bp product. The reaction occurred in a Perkin-Elmer Thermal Cycler under the following conditions: an initial denaturation at 95° C. for 5 min, followed by 40 cycles at 95° C. for 30s, 42° C. for 30s, 72° C. for 30s, with a final extension step at 72° C. for 7 min. GAPDH was used as a control to ensure equivalent loading and integrity of RNA.

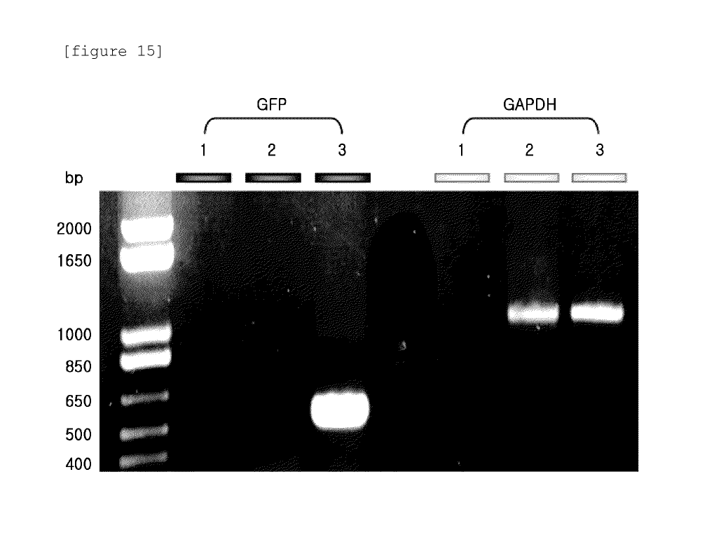

As shown in FIG. 15, the use of GFP specific primers in the PCR amplification resulted in a single 684 bp product from TRPC6^{DN} transfected rats. However, rats transfected with vector only (age-matched diabetic rat control) had no such band.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for treatment of erectile dysfunction, comprising a nucleotide sequence configured to express a polypeptide sequence for inhibiting a function of an ion channel that is involved in influx of ions into a tissue.

2. The composition according to claim 1, wherein the tissue is corporal smooth muscle.

3. The composition according to claim 1, wherein the ions are calcium ions (Ca²⁺).

4. The composition according to claim 1, wherein the ion channel is a TRPC6 ion channel.

5. The composition according to claim 1, wherein the the nucleotide sequence is defined by SEQ ID NO. 1.

6. A method of treating erectile dysfunction, the method comprising:
administering the composition of claim 1 to a person in need of such treatment.

7. A composition for treatment of erectile dysfunction, comprising a polypeptide configured to inhibit a function of an ion channel that is involved in influx of ions into a tissue.

8. The composition according to claim 7, wherein the tissue is corporal smooth muscle.

9. The composition according to claim 7, wherein the ions are calcium ions (Ca²⁺).

10. The composition according to claim 7, wherein the ion channel is a TRPC6 ion channel.

11. The composition according to claim 7, wherein the polypeptide is encoded by a nucleotide sequence of SEQ ID NO. 1.

12. A method of treating erectile dysfunction, the method comprising:
administering the composition of claim 7 to a person in need of such treatment.


SEQUENCE LISTING (10 sequences)

SEQ ID NO: 1 - DNA, 3261 bp, Artificial Sequence
  Dominant negative form of TRPC6
cgcctgtgccctctgcctgggagcctggggccgcctgtctgcgcggtccggatgcgctcaggtcaaggttcctttcgcggctgtctcccaagcccctaactagtgacttccactgtggcgggcagggaagccattggcagaacctagccagtcaggaatctgcatctcttccctcattatcctctccctggcattgctttgctcgggtccagctcagttggtgacgcggccccttctccccaggttgggatccacggaagcaggggtgcaggccggccaggcactgtgccatgagccagagcccgaggttcgtgacccggaggggcggctctctaaaggctgcccctggagccggcacccggcgcaacgagagccaggactatttgctgatggacgagctgggagacgacggctacccgcagctcccgctgccaccgtatggctactaccccagcttccggggtaatgaaaacagactgactcaccggcggcagacgattcttcgtgagaagggaagaaggttagctaatcgaggaccagcatacatgtttaatgatcattcaacaagcctgtctattgaggaagaacgctttctagatgcagttgaatatggcaacatcccagtggtctggaagatgctagaagagtgtcattccctcaatgttaactgtgtggattacatgggccagaatgccctacagctggctgtggccaatgagcacttggaaatcacagagctgctactcaagaaggaaaacttgtctcgagttggggatgctttacttttagccattagtaaaggttatgtacggattgtggaggcaatcctcaaccatccatcttttgctgaaggcaaaaggttagcgacaagccccagccagtctgaacttcagcaagatgacttttatgcctatgatgaagatgggacgcggttctcccatgatgtgactccaatcattctcgctgcacattgccaggaatatgaaattgtgcataccctcctgagaaagggtgcccggattgagcggcctcatgattacttctgcaagtgtacagaatgcagccagaagcagaagcatgattccttcagccactctagatccaggatcaatgcatacaaaggtctggcaagtccagcatacctgtcattgtccagtgaagatccagtcatgactgctttagaacttagcaatgagctggcagtgcttgccaacattgagaaagagttcaagaatgactacaggaagctgtctatgcagtgcaaggatttcgttgttggtctcttggacctctgcagaaacacagaggaagtggaggccatcctgaatggggatgcagagactcgccagcccggggacttcggccgtccaaatctcagccgtttaaaacttgctattaaggatgaagtaaaaaaatttgtggctcatccaaactgtcagcaacagctcctgtccatatggtatgagaacctctctggtttacggcagcagaccatggcagtgaagttcctcgtggtccttgctgttgccattggattgcccttcctggctctcatatactggtgtgctccttgcagcaagatggggaagatattgccgagaccgttcatgaagtttgtagcacacgcagcctccttcaccattttcctggggctgctcgtcatgaatgcagctgacagatttgaaggcaccaagctcctccctaatgaaaccagcacagataatgcaaggcagctgttcaggatgaaaacatcctgtttctcatggatggagatgctcattatatcctgggtaataggcatgatatgggctgaatgtaaagaaatctggactcaaggccccaaagaatacttatttgagttgtggaatatgcttgactttggaatgctggcaatctttgcagcatcattcattgcaagatttatggcgttctggcatgcatccaaagctcagagcatcattgatgcaaatgatactttaaaggatttgacaaaagtcacactgggggacaacgttaaatactacaatctggccaggataaagtgggaccctactgatcctcagatcatctctgaaggtctttatgcaatcgctgtggttttaagtttctccagaatagcttacattttaccagcaaatgaaagctttggacctctgcagatttcacttggaagaacagtgaaagatatcttcaaattcatggtcatattcatcatggtgtttgtagcctttatgattggaatgttcaacctttactcctactacattggcgcaaaacagaatgaagcattcacaacagttgaggaaagttttaagacagcgttctgggctatctttggtctttctgaagtgaagtcagtggtcattaactacaatcacaagttcattgaaaacatcggctacgttctgtatggtgtctataatgtcacaatggtcattgttttgctaaatatgttaattgcgatgatcaatagttcattccaggaaattgaggatgatgcggacgtggagtggaagtttgcaagggccaaattgtggttttcctactttgaggaggggagaacacttcctgtccccttcaatcttgtaccaagtccaaaatccttgctttatctcctattgaaatttaagaaatggatgtgtgagctcatccagggtcaaaagcaaggcttccaagaagatgcagagatgaacaagagaaatgaagaaaagaaatttggaatttcaggaagtcacgaagacctttcaaaattttcacttgacaaaaatcagttggcacacaacaaacaatcaagtacaaggagctcagaagattatcatttaaatagtttcagtaaccctccaagacaatatcagaaaatcatgaagagactcattaaaagatatgtattgcaggcccagattgataaggagagcgatgaggtgaatgaaggggaattgaaggaaattaagcaagacatctcaagtctccgttatgaactccttgaagagaaatcacagaactcagaagacctagcagagctcattagaaaactcggggagagactgtcgttagagccaaagctggaggaaagccgcagatagagcagagcccctcagaagtgcatatttatttctccacttgaagccatattattttctgacttatttttttaagtgtcaatgataaaaagtatgttaactgataacttggatcatttagagtcctaatatcaagctttttgggagattaaattgcattgctgagggctaacaattgctg

SEQ ID NO: 2 - DNA, 3261 bp, Mus musculus
  gene (1)..(3261): Wild type TRPC6
cgcctgtgccctctgcctgggagcctggggccgcctgtctgcgcggtccggatgcgctcaggtcaaggttcctttcgcggctgtctcccaagcccctaactagtgacttccactgtggcgggcagggaagccattggcagaacctagccagtcaggaatctgcatctcttccctcattatcctctccctggcattgctttgctcgggtccagctcagttggtgacgcggccccttctccccaggttgggatccacggaagcaggggtgcaggccggccaggcactgtgccatgagccagagcccgaggttcgtgacccggaggggcggctctctaaaggctgcccctggagccggcacccggcgcaacgagagccaggactatttgctgatggacgagctgggagacgacggctacccgcagctcccgctgccaccgtatggctactaccccagcttccggggtaatgaaaacagactgactcaccggcggcagacgattcttcgtgagaagggaagaaggttagctaatcgaggaccagcatacatgtttaatgatcattcaacaagcctgtctattgaggaagaacgctttctagatgcagttgaatatggcaacatcccagtggtctggaagatgctagaagagtgtcattccctcaatgttaactgtgtggattacatgggccagaatgccctacagctggctgtggccaatgagcacttggaaatcacagagctgctactcaagaaggaaaacttgtctcgagttggggatgctttacttttagccattagtaaaggttatgtacggattgtggaggcaatcctcaaccatccatcttttgctgaaggcaaaaggttagcgacaagccccagccagtctgaacttcagcaagatgacttttatgcctatgatgaagatgggacgcggttctcccatgatgtgactccaatcattctcgctgcacattgccaggaatatgaaattgtgcataccctcctgagaaagggtgcccggattgagcggcctcatgattacttctgcaagtgtacagaatgcagccagaagcagaagcatgattccttcagccactctagatccaggatcaatgcatacaaaggtctggcaagtccagcatacctgtcattgtccagtgaagatccagtcatgactgctttagaacttagcaatgagctggcagtgcttgccaacattgagaaagagttcaagaatgactacaggaagctgtctatgcagtgcaaggatttcgttgttggtctcttggacctctgcagaaacacagaggaagtggaggccatcctgaatggggatgcagagactcgccagcccggggacttcggccgtccaaatctcagccgtttaaaacttgctattaaggatgaagtaaaaaaatttgtggctcatccaaactgtcagcaacagctcctgtccatatggtatgagaacctctctggtttacggcagcagaccatggcagtgaagttcctcgtggtccttgctgttgccattggattgcccttcctggctctcatatactggtgtgctccttgcagcaagatggggaagatattgccgagaccgttcatgaagtttgtagcacacgcagcctccttcaccattttcctggggctgctcgtcatgaatgcagctgacagatttgaaggcaccaagctcctccctaatgaaaccagcacagataatgcaaggcagctgttcaggatgaaaacatcctgtttctcatggatggagatgctcattatatcctgggtaataggcatgatatgggctgaatgtaaagaaatctggactcaaggccccaaagaatacttatttgagttgtggaatatgcttgactttggaatgctggcaatctttgcagcatcattcattgcaagatttatggcgttctggcatgcatccaaagctcagagcatcattgatgcaaatgatactttaaaggatttgacaaaagtcacactgggggacaacgttaaatactacaatctggccaggataaagtgggaccctactgatcctcagatcatctctgaaggtctttatgcaatcgctgtggttttaagtttctccagaatagcttacattttaccagcaaatgaaagctttggacctctgcagatttcacttggaagaacagtgaaagatatcttcaaattcatggtcatattcatcatggtgtttgtagcctttatgattggaatgttcaacctttactcctactacattggcgcaaaacagaatgaagcattcacaacagttgaggaaagttttaagacactgttctgggctatctttggtctttctgaagtgaagtcagtggtcattaactacaatcacaagttcattgaaaacatcggctacgttctgtatggtgtctataatgtcacaatggtcattgttttgctaaatatgttaattgcgatgatcaatagttcattccaggaaattgaggatgatgcggacgtggagtggaagtttgcaagggccaaattgtggttttcctactttgaggaggggagaacacttcctgtccccttcaatcttgtaccaagtccaaaatccttgctttatctcctattgaaatttaagaaatggatgtgtgagctcatccagggtcaaaagcaaggcttccaagaagatgcagagatgaacaagagaaatgaagaaaagaaatttggaatttcaggaagtcacgaagacctttcaaaattttcacttgacaaaaatcagttggcacacaacaaacaatcaagtacaaggagctcagaagattatcatttaaatagtttcagtaaccctccaagacaatatcagaaaatcatgaagagactcattaaaagatatgtattgcaggcccagattgataaggagagcgatgaggtgaatgaaggggaattgaaggaaattaagcaagacatctcaagtctccgttatgaactccttgaagagaaatcacagaactcagaagacctagcagagctcattagaaaactcggggagagactgtcgttagagccaaagctggaggaaagccgcagatagagcagagcccctcagaagtgcatatttatttctccacttgaagccatattattttctgacttatttttttaagtgtcaatgataaaaagtatgttaactgataacttggatcatttagagtcctaatatcaagctttttgggagattaaattgcattgctgagggctaacaattgctg

SEQ ID NO: 3 - DNA, 22 bp, Artificial Sequence
  Forward primer for TRPC6
gtgccaagtccaaagtccctgc

SEQ ID NO: 4 - DNA, 20 bp, Artificial Sequence
  Reverse primer for TRPC6
ctgggcctgcagtacgtatc

SEQ ID NO: 5 - DNA, 21 bp, Artificial Sequence
  Forward primer for GADPH
atagacaagatggtgaaggtc

SEQ ID NO: 6 - DNA, 21 bp, Artificial Sequence
  Reverse primer for GADPH
tactccttggaggccatgtag

SEQ ID NO: 7 - DNA, 20 bp, Artificial Sequence
  Forward primer for GFP in the first round of amplification
ctgaccctgaagttcatctg

SEQ ID NO: 8 - DNA, 18 bp, Artificial Sequence
  Reverse primer for GFP in the first round of amplification
taccgtcgactgcagaat

SEQ ID NO: 9 - DNA, 19 bp, Artificial Sequence
  Forward primer for GFP in the second round of amplification
tcgtgaccaccctgaccta

SEQ ID NO: 10 - DNA, 20 bp, Artificial Sequence
  Reverse primer for GFP in the second round of amplification
cttgtacagctcgtccatgc